(19) United States
(12) Patent Application Publication
(10) Pub. No.: US 2025/0375765 A1
(43) Pub. Date: Dec. 11, 2025

(54) LAB BENCH ARRAY, SYSTEM AND METHODOLOGY FOR PLANT CELLS

(71) Applicant: INTERNATIONAL BUSINESS MACHINES CORPORATION, ARMONK, NY (US)

(72) Inventors: Frank Robert Libsch, White Plains, NY (US); Venkat Kanagraj Balagurusamy, Airmont, NY (US)

(21) Appl. No.: 18/740,476

(22) Filed: Jun. 11, 2024

(51) Int. Cl. B01L 3/00; G01N 27/22; G01N 33/00

(52) U.S. Cl. CPC B01L 3/502715; G01N 27/226; G01N 33/0098; B01L 2200/027; B01L 2200/0647; B01L 2300/0645; B01L 2300/12; B01L 2300/1805

(57) ABSTRACT

A structure for sensing material movement includes a set of first conductive electrodes in a top substrate and a set of second conductive electrodes in a bottom substrate positioned adjacent to and spaced apart from the set of first conductive electrodes in a top substrate. A microfluidic chamber is defined within a space between the top substrate and the bottom substrate. A first set of through vias in the top substrate connect the set of first conductive electrodes to a set of first signal lines on a top side of the top substrate. A second set of through vias in the bottom substrate connect the set of second conductive electrodes to a set of second signal lines on the bottom side of the bottom substrate.

### BACKGROUND

The present disclosure generally relates to systems and methods for identifying reproductive quality and growth potential of plant cells, and more particularly, to a portable lab bench array that can be used, with built-in environment controls, to determine reproductive quality and growth potential of plant cells.

Plant cells can range from 20 to 100 microns in size. It is known that the organelles in plant cells exhibit streaming motion (movement in near straight-line like an object moving in a stream) which helps as a transport mechanism for carrying nutrients to different parts of cells which is vital for the metabolic activities. It has also been associated with the growth potential of plants at the cellular level. As the cytoplasm in plant cells range from 4 to 10 microns, one could observe this motion easily under the microscope.

In addition, mitochondria which are much smaller, about 0.2 micron, also exhibit such motion which is somewhat difficult to observe under the microscope due partly to their small size and also transparent colorless nature. The movement of the motor proteins (Mysosin) attached to the organelles along the actin microfilaments is thought to be the key mechanism for the transport.

### SUMMARY

In one embodiment, a system and method are described for portable technology that can identify reproductive quality and growth potential of cells in plant saplings. Observation at the cellular levels to monitor organelle motion is not suited to microscopic observation. Instrumentation that may permit such observations are cost restrictive and unscalable to be deployable over a number of locations. Aspects of the present disclosure can be used for observing with a microscope with transparent electrodes and can explore a larger parameter space of environmental variables in a cost-effective and time-effective way.

In one embodiment, a structure for sensing material movement includes a set of first conductive electrodes in a top substrate and a set of second conductive electrodes in a bottom substrate positioned adjacent to and spaced apart from the set of first conductive electrodes in a top substrate. A microfluidic chamber is defined within a space between the top substrate and the bottom substrate. A first set of through vias in the top substrate connect the set of first conductive electrodes to a set of first signal lines on a top side of the top substrate. A second set of through vias in the bottom substrate connect the set of second conductive electrodes to a set of second signal lines on the bottom side of the bottom substrate.

In another embodiment, a structure for sensing material movement includes a set of first conductive electrodes in a top substrate and a set of second conductive electrodes in a bottom substrate positioned adjacent to and spaced apart from the set of first conductive electrodes in a top substrate. A microfluidic chamber is defined within a space between the top substrate and the bottom substrate. A first set of through vias in the top substrate connect the set of first conductive electrodes to a set of first signal lines on a top side of the top substrate. A second set of through vias in the bottom substrate connect the set of second conductive electrodes to a set of second signal lines on the bottom side of the bottom substrate. A heating element is disposed along at least a portion of a bottom surface of the top substrate and at least a portion of a top surface of the bottom substrate. An illumination device is operable to deliver a user-selectable wavelength of light into the microfluidic chamber.

In another embodiment, a method for sensing movement within plant cells includes providing a solution containing the plant cells into the microfluidic chamber of a sensor structure, such as the structure described above. The capacitance of the microfluidic channel between opposing ones of the first set of electrodes and the second set of electrodes is measured and changes in the capacitance are correlated to movement within plant cells.

These and other features will become apparent from the following detailed description of illustrative embodiments thereof, which is to be read in connection with the accompanying drawings.

### BRIEF DESCRIPTION OF THE DRAWINGS

The drawings are of illustrative embodiments. They do not illustrate all embodiments. Other embodiments may be used in addition or instead. Details that may be apparent or unnecessary may be omitted to save space or for more effective illustration. Some embodiments may be practiced with additional components or steps and/or without all the components or steps that are illustrated. When the same numeral appears in different drawings, it refers to the same or like components or steps.

FIG. 1 shows a sensor for sensing movement within a microfluidic flow, consistent with an illustrative embodiment;

FIG. 2 shows a cross-sectional view taken along line II-II of FIG. 1;

FIG. 3 shows top view of a first transparent substrate of the sensor of FIG. 1, detailing in-line electrodes and showing a resistive heater element, consistent with an illustrative embodiment;

FIG. 4 is a cross-sectional view taken along line IV-IV of FIG. 3;

FIG. 5 is a cross-sectional view taken along line V-V of FIG. 3;

FIG. 6 is a top view of a second transparent substrate of the sensor of FIG. 1, detailing in-line electrodes and showing a resistive heater element, consistent with an illustrative embodiment;

FIG. 7 is a cross-sectional view taken along line VII-VII of FIG. 6;

FIG. 8 is a cross-sectional view taken along line VIII-VIII of FIG. 6;

FIG. 9 is a top view of the first and second substrates of FIGS. 3 and 6, detailing in-line electrodes and showing ground/shield materials, consistent with an illustrative embodiment;

FIG. 10 is a cross-sectional view taken along line X-X of FIG. 9;

FIG. 11 is a cross-sectional view taken along line XI-XI of FIG. 9;

FIG. 12 is a top view of the first and second substrates of FIGS. 3 and 6, detailing in-line electrodes and showing a thermistor element, (a resistive heater element may be of the same pattern and, in this embodiment, is on the second substrate), consistent with an illustrative embodiment;

FIG. 13 is a cross-sectional view taken along line XIII-XIII of FIG. 12;

FIG. 14 is a cross-sectional view taken along line XIV-XIV of FIG. 12;

FIG. 15 is a top view showing, after assembly of the first and second substrates of FIGS. 3 and 6, in-line electrodes, a multiwavelength illumination element, a thermistor, and a resistive heating element, consistent with an illustrative embodiment;

FIG. 16 is a cross-sectional view taken along line XVI-XVI of FIG. 15;

FIG. 17 is a top view showing, after assembly of the first and second substrates of FIGS. 3 and 6, in-line electrodes, a multiwavelength illumination element, a thermistor, a resistive heating element, and a microfluidic seal, consistent with an illustrative embodiment;

FIG. 18 is a cross-sectional view taken along line XVIII-XVIII of FIG. 17;

FIGS. 19A and 19B illustrate a flow chart illustrating a method, consistent with an illustrative embodiment, where FIG. 19B is a continuation of FIG. 19A, with the circled characters (A, B, C, and D) of FIG. 19A connecting with the corresponding circled characters (A′, B′, C′, and D′) of FIG. 19B; and

FIG. 20 is a functional block diagram illustration of a computer hardware platform that can be used to implement the method for sensing plant cell movements, consistent with an illustrative embodiment.

### DETAILED DESCRIPTION

In the following detailed description, numerous specific details are set forth by way of examples to provide a thorough understanding of the relevant teachings. However, it should be apparent that the present teachings may be practiced without such details. In other instances, well-known methods, procedures, components, and/or circuitry have been described at a relatively high-level, without detail, to avoid unnecessarily obscuring aspects of the present teachings.

As described in greater detail below, aspects of the present disclosure provide systems and methods that can provide a portable lab bench array that can detect movement with a plant cell.

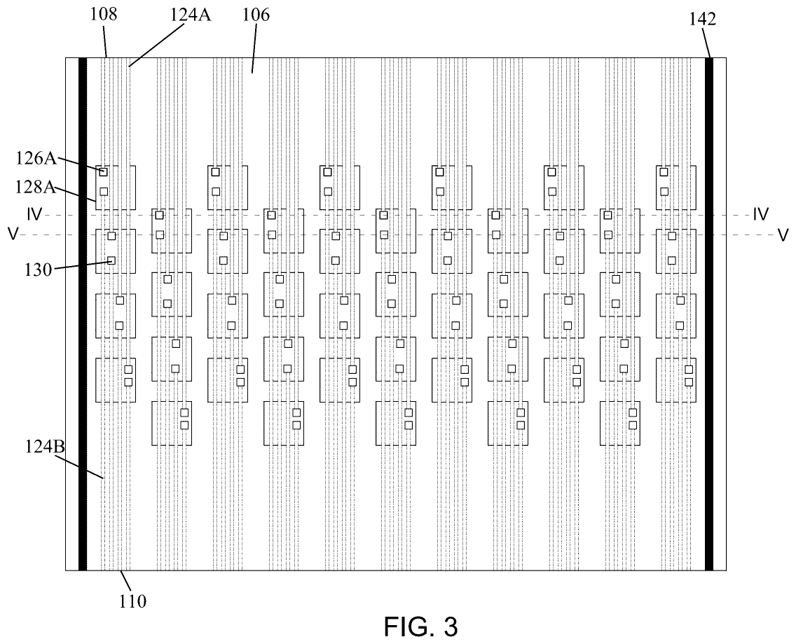

As used herein, terms such as “upper,” “lower,” “horizontal” and “vertical” are used to describe directions relative to the images shown in the Figures. Thus, in FIG. 3, edge 108 may be described as an upper edge, while edge 110 may be described as a lower edge, and the conductive electrodes 128A, shown in FIG. 3, may be vertically aligned, while every other column of conductive electrodes 128A may be horizontally aligned.

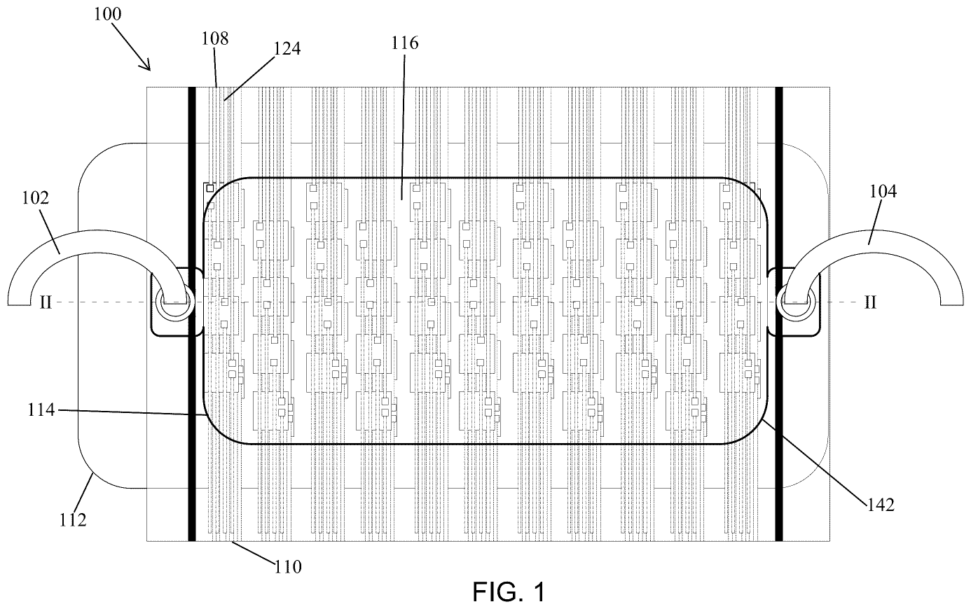

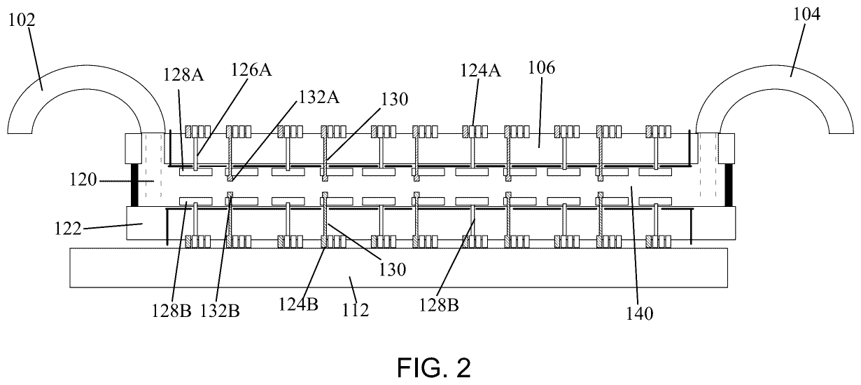

Referring to FIGS. 1 and 2, an assembled sensor 100 is illustrated. Each component of the sensor 100 will be later described in greater detail below. The sensor 100 can include a top substrate 106 and a bottom substrate 122 that are positioned with a space 140 therebetween. In some embodiments, the top substrate 106 is disposed parallel to the bottom substrate 122. The space 140 can define a microfluidic chamber 116 for sensing material movement, such as movement of components within a cell, such as a plant cell. A dielectric spacer 142 can be used to space the top substrate 106 from the bottom substrate 122. The dielectric spacer may be independent of the gasket 120. Other methods of separating the top and bottom substrates at precise heights may be formed with spacer balls of a particular diameter and made of such inert material as polystyrene. Yet another method may be to have the gasket 120 height determine the separation distance of the top and bottom substrates, and hence, microfluidic channel height.

In FIG. 1, for clarity the ground/shield, resistive heater and thermistor, as described in greater detail below, are not shown and the top and bottom substrates 106, 122 are shown as transparent and slightly displaced.

A gasket 120 can be disposed between the top substrate 106 and the bottom substrate 122 to create side boundaries for the microfluidic chamber 116. There are several techniques for forming the gasket 120, such as heat reflowed solder gasket, where the gasket may be formed as mirror twins of a solder material, one gasket on the top substrate 106, and one gasket on the bottom substrate 122, and the two substrates are mated and heated to reflow the solder gasket. There are various low and high temperature solders and corresponding metal systems, which include copper with SAC (tin with small percentages of aluminum and copper), nickel with SAC, gold/tin interlayers, nickel/gold with tin, bismuth alloys, and the like. Another method for forming the gasket 114 includes mechanical pressure interlocking lid sealing, where one substrate contains a double hump camel gasket shape, and the other substrate contains a single hump gasket shape, where upon mating, the double hump gasket is tension forced to straddle and lock onto the single hump gasket, which can be fabricated during the substrate microfabrication process. Another method for forming the gasket 114 includes forming a chemical epoxy gasket which may be cured by any one or several methods: thermal, pressure, and/or optical.

In some embodiments, an inlet port 102 may fluidly connect to a through hole 120 through the top substrate 106 to provide a fluid connection between an exterior of the sensor 100 and the microfluidic chamber 116. An outlet port 104 may be similarly created, typically opposite the inlet port 102, as shown in FIGS. 1 and 2. While the use of the inlet port 102 and the outlet port 104 provides one example of a method to introduce a fluid to be analyzed into the microfluidic chamber 116, other methods may also be used, such as connecting the outlet port 104 to a vacuum, precise metering of copolymer or buffer volume onto the bottom substrate 122 before assembling the top and bottom substrates 106, 122, one (or more) port(s) that are first subjected to vacuum and then backfilled with solution, or the like. Further, while the figures show the inlet and outlet ports 102, 104 disposed on opposite ends of the sensor 100, various location arrangements may be used, such as two inlet ports on opposite sides of the microfluid chamber and an outlet port at a central region thereof, or the like.

As discussed in greater detail below, the top substrate 106 can include a plurality of signal probe lines 126A that extend through the top substrate 106 to interconnect probe lines 124, disposed on a top of the top substrate 106 to conductive electrodes 128A disposed on a bottom side of the top substrate 106. Similarly, the bottom substrate 122 can include a plurality of signal probe lines 126B that extend through the bottom substrate 122 to interconnect probe lines 124, disposed on a bottom of the bottom substrate 122 to conductive electrodes 128B disposed on a top side of the bottom substrate 122. As can be seen, the conductive electrodes 128A, 128B can be formed within the microfluidic chamber 116, facing each other.

The set of conductive electrodes 128A of the top substrate 106 and the set of conductive electrodes 128A of the bottom substrate 122 can form a one or more dimensional array of electrodes, as illustrated in FIG. 1, for example. The top substrate 106 and bottom substrate 122 can be formed as electrically insulating substrates. In some embodiments, the electrically insulating substrates are one of glass, plastic, or organic printed circuit boards. Each of the conductive electrodes 128A may be electrically insulated from each other. In some embodiments, each conductive electrode 128A, 128B are formed from transparent conductive films, such as indium tin oxide (ITO), fluorine doped tine oxide (FTO), or doped zinc oxide.

Further, as discussed in greater detail below, coax lines 130 can extend through the top and bottom substrates 106, 122 to connect the probe lines 124 to sensors 132A, 132B disposed in the microfluidic chamber 116. The coax lines 130 may be useful for sensors 132A, 132B that may require shielding the signals from the sensors 132A, 132B.

Referring now to FIGS. 3 through 5, a top view and cross-sectional views of the top substrate 106 of the sensor 100 of FIG. 1 are shown. A set of upper probe lines 124A, disposed on a top side of the top substrate 106, can extend from a first edge 108 of the top substrate 106 and can interconnect, via signal probe lines 126A, the upper probe lines 124A to conductive electrodes 128A disposed on the bottom side of the top substrate 106. A set of lower probe lines 124B, also disposed on a top side of the top substrate 106, can extend from a second edge 110, opposite the first edge 108, of the top substrate 106 and can interconnect, via coax lines 130, the lower probe lines 124B to sensors 132A disposed within the microfluidic chamber 116. Connectors (not shown) may be used to electrically connect to the upper and lower probe lines 124A, 124B at the first edge 108 and the second edge 110, respectively. The connectors may provide signals from the upper and lower probe lines 124A, 124B to a computing device for training or using deep learning models, as described in greater detail below, for sensing movement, such as organelle movement within a plant cell.

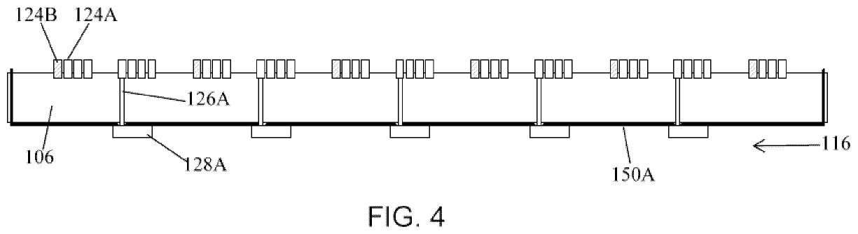

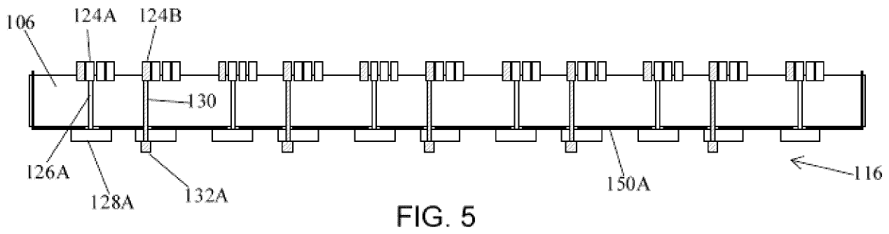

A resistive heating element 150A may be disposed at least along a portion of the bottom side of the top substrate 106, as best illustrated in FIGS. 4 and 5. The resistive heating element 150A may be used to control a temperature of the fluid within the microfluidic chamber 116.

While the figures show the sensors 132A disposed within each of the conductive electrodes 128A, it should be understood that the sensors 132A may be placed at various locations within the microfluidic chamber 116, depending on the particular application.

While the figures show four conductive electrodes 128A formed along each column of the top substrate 106, with four lines of each of the upper probe lines 124A and the lower probe lines 124B, it should be understood that a greater or fewer number of conductive electrodes 128A may be disposed in each column. Further, while the figures show the conductive electrodes 128A disposed in a staggered arrangement from one column to another, it should be understood that other arrangements may be used, such as both vertical and horizontal alignment of the conductive electrodes 128A.

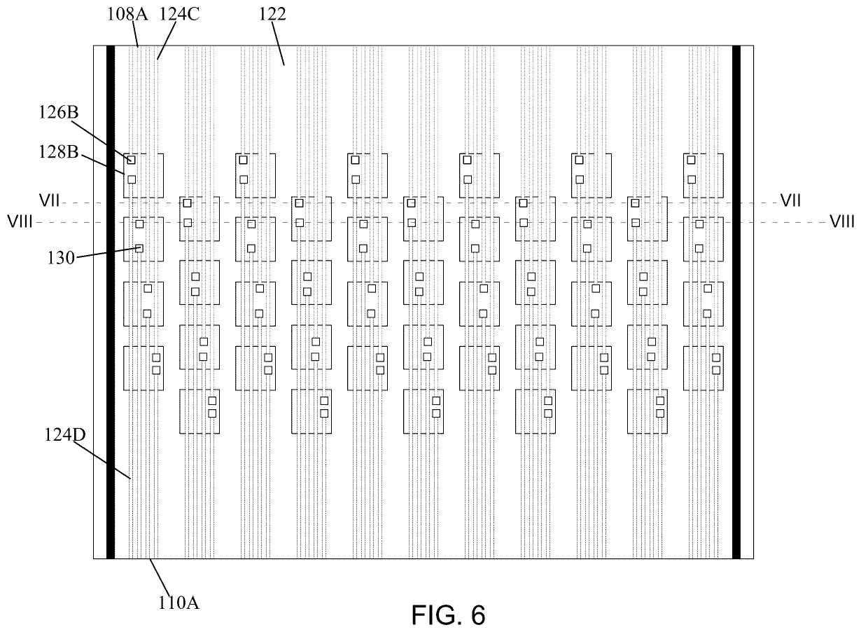

Referring now to FIGS. 6 through 8, a bottom view and cross-sectional views of the bottom substrate 122 of the sensor 100 of FIG. 1 are shown. A set of upper probe lines 124C, disposed on a bottom side of the bottom substrate 122, can extend from a first edge 108A of the bottom substrate 122 and can interconnect, via signal probe lines 126B, the upper probe lines 124C to conductive electrodes 128B disposed on the top side of the bottom substrate 122. A set of lower probe lines 124D, also disposed on a bottom side of the bottom substrate 122, can extend from a second edge 110A, opposite the first edge 108A, of the bottom substrate 122 and can interconnect, via coax lines 130, the lower probe lines 124D to sensors 132B disposed within the microfluidic chamber 116.

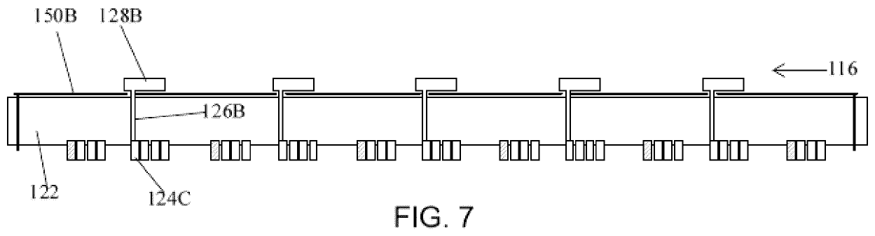

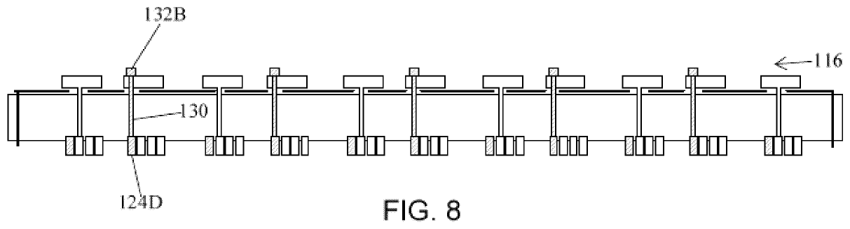

A resistive heating element 150B may be disposed at least along a portion of the top side of the bottom substrate 122, as best illustrated in FIGS. 7 and 8. The resistive heating element 150B may be used to control a temperature of the fluid within the microfluidic chamber 116.

While the figures show the sensors 132B disposed within each of the conductive electrodes 128B, it should be understood that the sensors 132B may be placed at various locations within the microfluidic chamber 116, depending on the particular application.

While the figures show four conductive electrodes 128B formed along each column of the bottom substrate 122, with four lines of each of the upper probe lines 124C and the lower probe lines 124D, it should be understood that a greater or fewer number of conductive electrodes 128B may be disposed in each column (along with the associated number of upper and lower probe lines 124C, 124D). Further, while the figures show the conductive electrodes 128B disposed in a staggered arrangement from one column to another, it should be understood that other arrangements may be used, such as both vertical and horizontal alignment of the conductive electrodes 128B.

As can be seen in FIGS. 1 and 2, the conductive electrodes 128A of the top substrate 106 may vertically align with the conductive electrodes 128B of the bottom substrate 122. In some embodiments, the conductive electrodes 128A, 128B may be used to measure capacitance or reactance between opposing pairs of conductive electrodes 128A, 128B.

While FIGS. 1 through 8 show a particular arrangement, orientation, and density, it should be understood that the size, arrangement, and number of conductive electrodes within the microfluidic chamber may vary depending on application. In some embodiments, a single plant cell can be disposed between from one to ten sets of conductive electrodes 128A, 128B, for example. For use in measuring movement within a plant cell, changes in capacitance between the conductive electrodes 128A, 128B may be determinative of the movement within the cell.

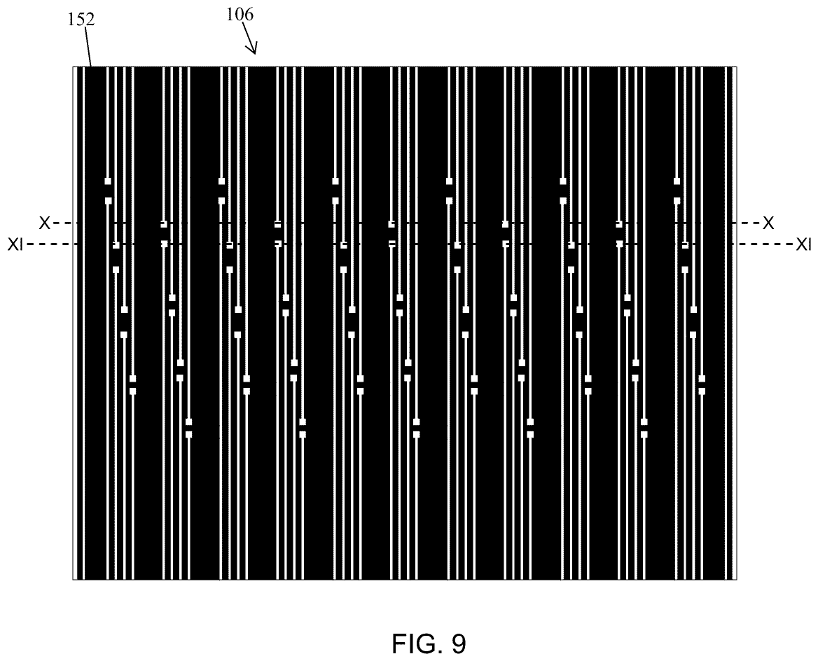

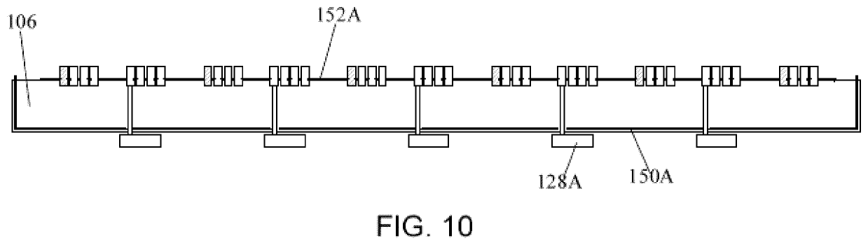

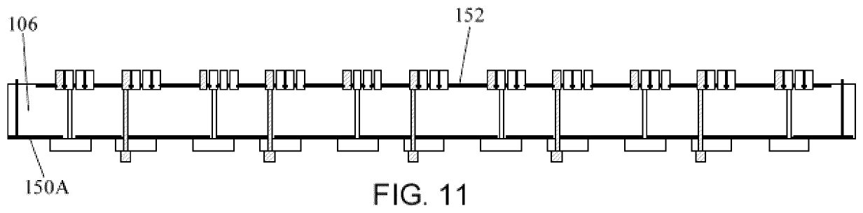

Referring now to FIGS. 9 through 11, the top substrate 106 is shown, detailing the ground/shield pattern 152 that may be applied thereto. The ground/shield pattern 152 may be formed from the same metal layer as probe layer metal since the ground/shield pattern 152 does not intersect with probe metallization pattern. In some embodiments, the ground/shield pattern 152 may be formed from a metal different from that forming the probe layer. While the top substrate 106 is shown, with the ground/shield pattern 152 applied to a top side thereto, a similar ground/shield pattern can be used on the bottom side of the bottom substrate 122. The ground/shield pattern 152 can be formed from a transparent metal such as indium tin oxide (ITO), or the like, if transparency is desired.

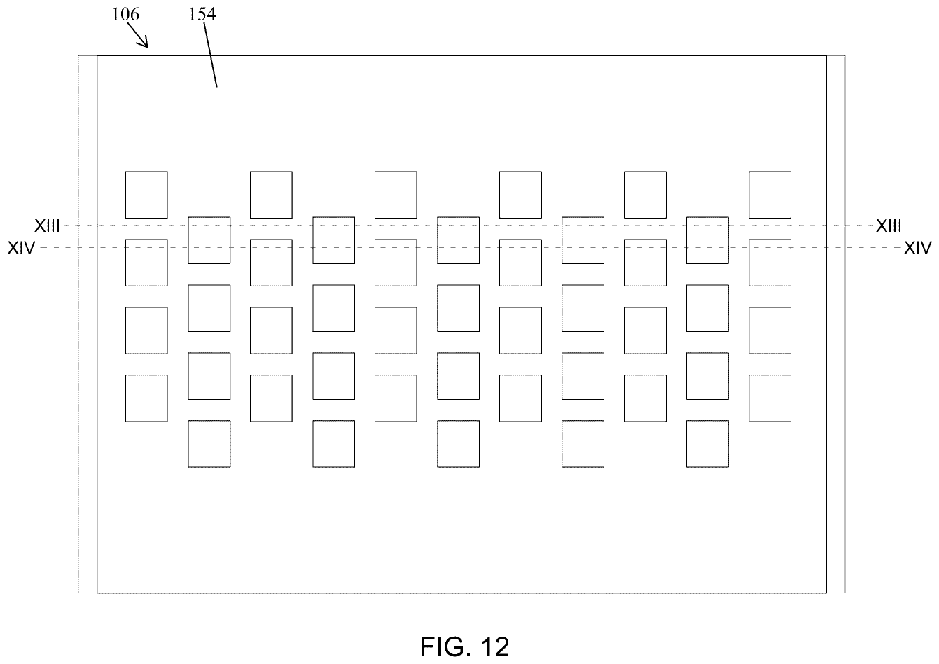

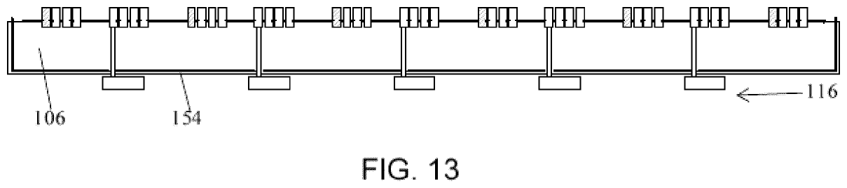

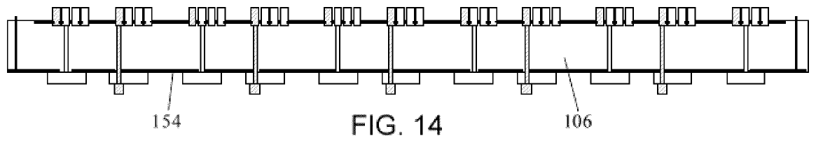

Referring now to FIGS. 12 through 14, a thermistor element 154 can be disposed on a bottom side of the top substrate 106 and, similarly, on the top side of the bottom substrate 122. The thermistor element 154 may be used to measure a temperature within the microfluidic chamber 116. The resistive heater element may be of the same pattern to adjust a temperature within the microfluidic chamber 116. The thermistor element 154, as well as the resistive heater element, can be formed from a transparent metal such as indium tin oxide (ITO), or the like, if transparency is desired.

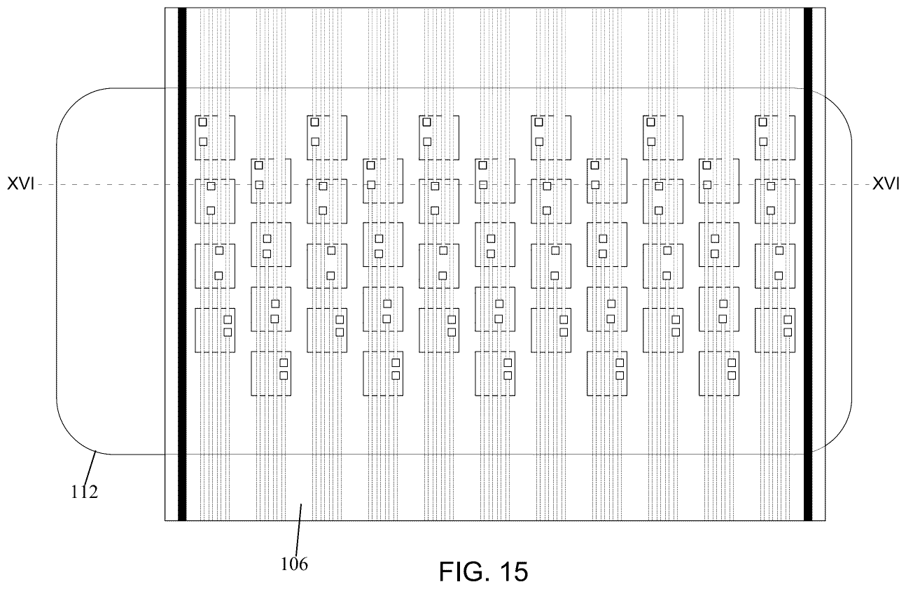

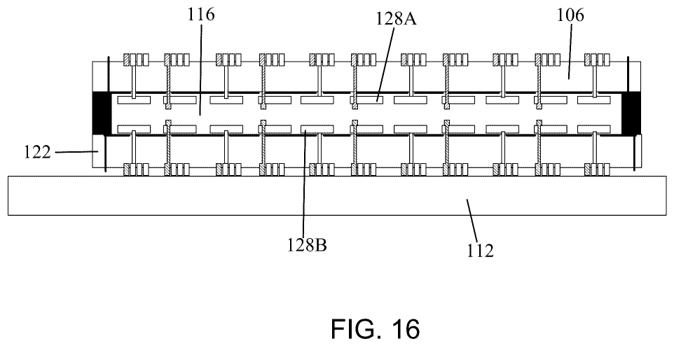

Referring now to FIGS. 15 and 16, the assembled sensor is shown, leaving out the inlet port, outlet port, ground/shield pattern, resistive heating element, and thermistor element for clarity. Once the top substrate 106 is connected with the bottom substrate 122, forming the microfluidic chamber 116 therebetween, the assembly can be mounted on an illumination device 112 that can provide multiwavelength illumination to the microfluidic chamber 116. The illumination device 112 can be adjusted to change the wavelength to illuminate the microfluidic chamber 116, where measurements may be made to determine the effect of various types of illumination on plant cells.

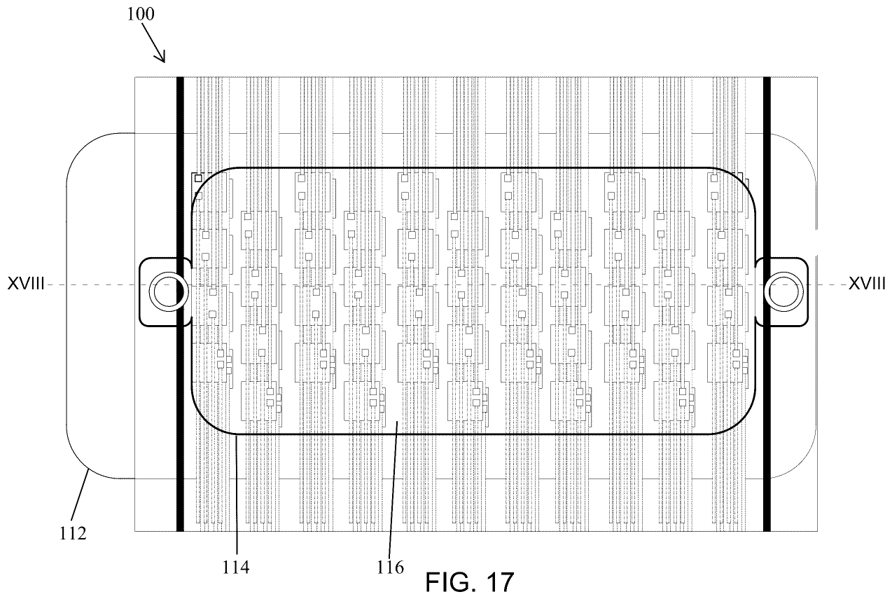

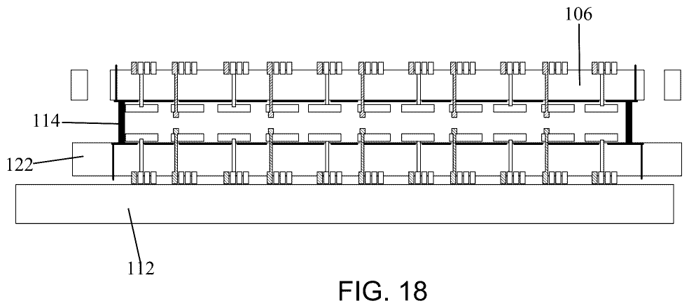

Referring to FIGS. 17 and 18, the gasket 114 is illustrated in the sensor 100, where the gasket 114 defines the microfluidic chamber 116. While a rectangular shaped gasket 114 is shown, it should be understood that the size and shape of the gasket 114 (and thus, the size and shape of the microfluidic chamber 116) may vary. Typically, the microfluid chamber 116 may be sized with a height from 1.5 to 5 times the height of a plant cell. The width and length of the microfluidic chamber 116 may be sized to be from 1 to 10 plant cells in length and width. Of course, the size may be smaller or larger, depending on the particular study, measurements, or the like.

### Example Process

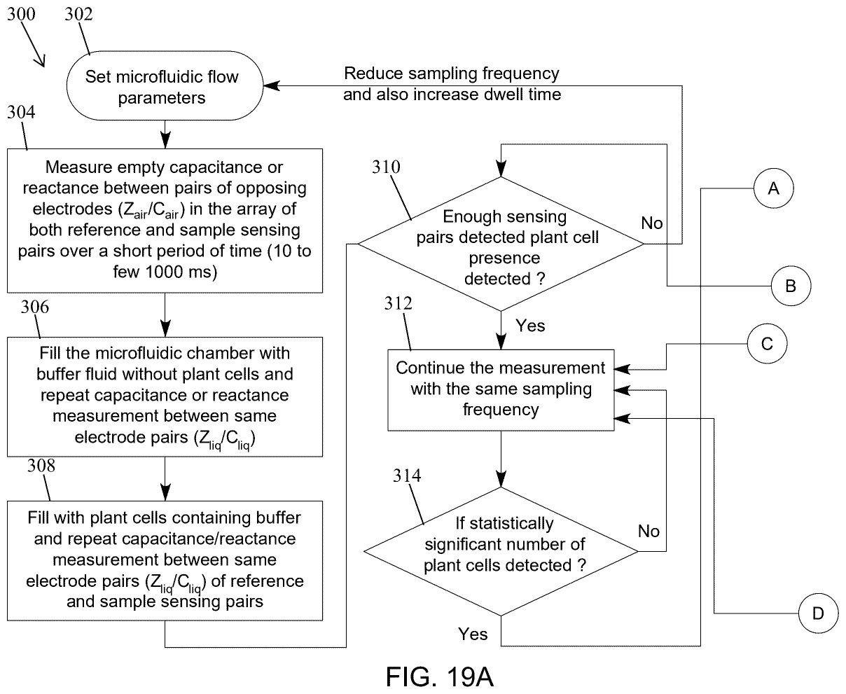

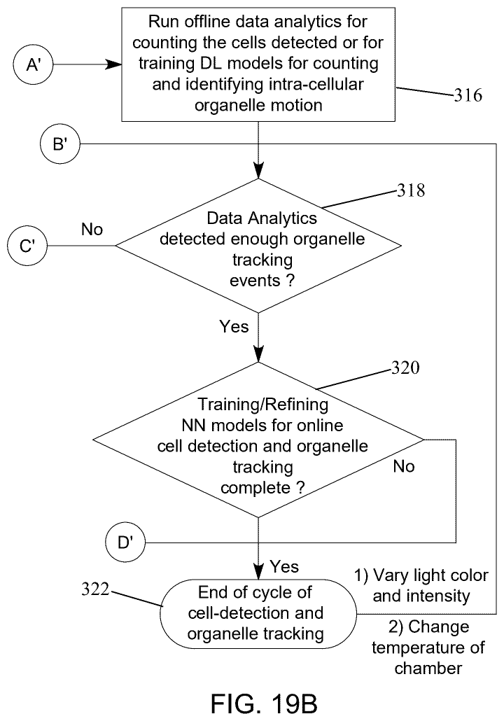

It may be helpful now to consider a high-level discussion of an example process. To that end, FIG. 19 presents an illustrative process related to the method for sensing plant cellular movement. Process 300 are illustrated as a collection of blocks, in a logical flowchart, which represents a sequence of operations that can be implemented in hardware, software, or a combination thereof. In the context of software, the blocks represent computer-executable instructions that, when executed by one or more processors, perform the recited operations. Generally, computer-executable instructions may include routines, programs, objects, components, data structures, and the like that perform functions or implement abstract data types. In each process, the order in which the operations are described is not intended to be construed as a limitation, and any number of the described blocks can be combined in any order and/or performed in parallel to implement the process.

Referring to FIGS. 19A and 19B, block 302 of process 300, can include an act of receiving a setting of the microfluidic flow parameters. These parameters can include flow rate, flow volume, plant cell concentration, sampling frequency, dwell time, temperature, wavelength and/or intensity of lighting, or the like. At block 304, the capacitance or reactance between pairs of opposing electrodes is measured when the microfluidic chamber is empty. This can be performed over a short period of time, such as between 10 milliseconds (ms) and up to several thousand milliseconds.

At block 306, the microfluidic chamber can be filled with buffer fluid without plant cells therein. Capacitance and/or reactance can be measured between the same electrode pairs. At block 308, the microfluidic chamber can be filled with plant cells containing the same buffer used at block 306. Capacitance and/or reactance can be measured between the same electrode pairs.

At decision block 310, it can be determined if enough sensing pairs have detected the presence of plant cells. This can be determined, for example, between detecting a difference in the capacitance and/or reactance between blocks 306 and 308. If enough sensing pairs of opposing electrodes have not detected plant cells, then the sampling frequency can be reduced and/or the dwell time can be increased and the process can be repeated. If enough sensing pairs of opposing electrodes have detected plant cells, the process moves to block 312, where the measurements are continued at the same sampling frequency.

At decision block 314, it can be determined whether a statistically significant number of plant cells have been detected. This statistically significant number can be a predetermined number, set, for example, with the microfluidic flow parameters, or may be determined by the system, through deep learning, for example, as discussed in greater detail below. If a statistically significant number of plant cells have not been detected, then, at block 312, the measurements continue. If a statistically significant number of plant cells have been detected, then the process continues to block 316.

At block 316, offline data analytics can be run for counting the cells detected or for training deep learning models for counting and identifying intra-cellular organelle motion. During training of the deep learning models, the system may use specialized tools (such as non-portable, costly, conventional microscopes or other such tools) to detect intra-cellular organelle motion and correlate such motion to the changes in capacitance and/or reactance measured by the electrodes. Thus, during inference, a system and related trained deep learning algorithms can used where the data received from the electrodes can be used to detect and determine intra-cellular organelle motion.

At block 318, a decision block can determine whether data analytics have detected enough organelle tracking events. If not, the process reverts back to block 312 to continue with measurements. If enough organelle tracking events have been detected, then the process continues to decision block 320, where it is determined whether training/refining of neural network models for online cell detection and organelle tracking is complete. If so, then the process ends at block 322. If not, then the process can revert to block 312 to continue with measurements. At the end, in block 322, the system can optionally vary light color and/or intensity and can also change the temperature of the microfluidic chamber and the process can revert to block 310, and additional data can be taken under different illumination and/or temperature control. Such changes can be useful to detect whether different lighting and/or temperature may be beneficial for identifying reproductive quality and growth potential of plant cells.

### Example Computing Platform

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

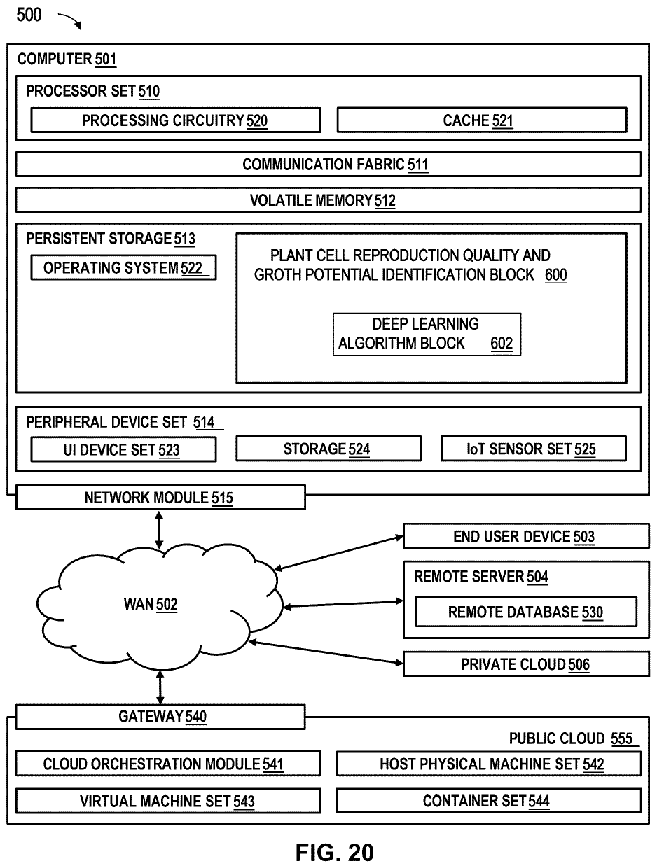

Referring to FIG. 20, computing environment 500 includes an example of an environment for the execution of at least some of the computer code involved in performing the inventive methods, including a plant cell reproductive quality and growth potential identification block 600, which can include a deep learning algorithm block 602, which, as discussed above, can be trained to correlate changes in measured capacitance and/or reactance to movement within an plant cell, such as intra-cellular organelle motion. In addition to block 600, computing environment 500 includes, for example, computer 501, wide area network (WAN) 502, end user device (EUD) 503, remote server 504, public cloud 505, and private cloud 506. In this embodiment, computer 501 includes processor set 510 (including processing circuitry 520 and cache 521), communication fabric 511, volatile memory 512, persistent storage 513 (including operating system 522 and block 600, as identified above), peripheral device set 514 (including user interface (UI) device set 523, storage 524, and Internet of Things (IoT) sensor set 525), and network module 515. Remote server 504 includes remote database 530. Public cloud 505 includes gateway 540, cloud orchestration module 541, host physical machine set 542, virtual machine set 543, and container set 544.

COMPUTER 501 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 530. As is well understood in the art of computer technology, and depending upon the technology, performance of a computer-implemented method may be distributed among multiple computers and/or between multiple locations. On the other hand, in this presentation of computing environment 500, detailed discussion is focused on a single computer, specifically computer 501, to keep the presentation as simple as possible. Computer 501 may be located in a cloud, even though it is not shown in a cloud in FIG. 5. On the other hand, computer 501 is not required to be in a cloud except to any extent as may be affirmatively indicated.

PROCESSOR SET 510 includes one, or more, computer processors of any type now known or to be developed in the future. Processing circuitry 520 may be distributed over multiple packages, for example, multiple, coordinated integrated circuit chips. Processing circuitry 520 may implement multiple processor threads and/or multiple processor cores. Cache 521 is memory that is located in the processor chip package(s) and is typically used for data or code that should be available for rapid access by the threads or cores running on processor set 510. Cache memories are typically organized into multiple levels depending upon relative proximity to the processing circuitry. Alternatively, some, or all, of the cache for the processor set may be located “off chip.” In some computing environments, processor set 510 may be designed for working with qubits and performing quantum computing.

Computer readable program instructions are typically loaded onto computer 501 to cause a series of operational steps to be performed by processor set 510 of computer 501 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 521 and the other storage media discussed below. The program instructions, and associated data, are accessed by processor set 510 to control and direct performance of the inventive methods. In computing environment 500, at least some of the instructions for performing the inventive methods may be stored in block 600 in persistent storage 513.

COMMUNICATION FABRIC 511 is the signal conduction path that allows the various components of computer 501 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 512 is any type of volatile memory now known or to be developed in the future. Examples include dynamic type random access memory (RAM) or static type RAM. Typically, volatile memory 512 is characterized by random access, but this is not required unless affirmatively indicated. In computer 501, the volatile memory 512 is located in a single package and is internal to computer 501, but, alternatively or additionally, the volatile memory may be distributed over multiple packages and/or located externally with respect to computer 501.

PERSISTENT STORAGE 513 is any form of non-volatile storage for computers that is now known or to be developed in the future. The non-volatility of this storage means that the stored data is maintained regardless of whether power is being supplied to computer 501 and/or directly to persistent storage 513. Persistent storage 513 may be a read only memory (ROM), but typically at least a portion of the persistent storage allows writing of data, deletion of data and re-writing of data. Some familiar forms of persistent storage include magnetic disks and solid state storage devices. Operating system 522 may take several forms, such as various known proprietary operating systems or open source Portable Operating System Interface-type operating systems that employ a kernel. The code included in block 600 typically includes at least some of the computer code involved in performing the inventive methods.

PERIPHERAL DEVICE SET 514 includes the set of peripheral devices of computer 501. Data communication connections between the peripheral devices and the other components of computer 501 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 523 may include components such as a display screen, speaker, microphone, wearable devices (such as goggles and smart watches), keyboard, mouse, printer, touchpad, game controllers, and haptic devices. Storage 524 is external storage, such as an external hard drive, or insertable storage, such as an SD card. Storage 524 may be persistent and/or volatile. In some embodiments, storage 524 may take the form of a quantum computing storage device for storing data in the form of qubits. In embodiments where computer 501 is required to have a large amount of storage (for example, where computer 501 locally stores and manages a large database) then this storage may be provided by peripheral storage devices designed for storing very large amounts of data, such as a storage area network (SAN) that is shared by multiple, geographically distributed computers. IoT sensor set 525 is made up of sensors that can be used in Internet of Things applications. For example, one sensor may be a thermometer and another sensor may be a motion detector.

NETWORK MODULE 515 is the collection of computer software, hardware, and firmware that allows computer 501 to communicate with other computers through WAN 502. Network module 515 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 515 are performed on the same physical hardware device. In other embodiments (for example, embodiments that utilize software-defined networking (SDN)), the control functions and the forwarding functions of network module 515 are performed on physically separate devices, such that the control functions manage several different network hardware devices. Computer readable program instructions for performing the inventive methods can typically be downloaded to computer 501 from an external computer or external storage device through a network adapter card or network interface included in network module 515.

WAN 502 is any wide area network (for example, the internet) capable of communicating computer data over non-local distances by any technology for communicating computer data, now known or to be developed in the future. In some embodiments, the WAN 502 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 503 is any computer system that is used and controlled by an end user (for example, a customer of an enterprise that operates computer 501) and may take any of the forms discussed above in connection with computer 501. EUD 503 typically receives helpful and useful data from the operations of computer 501. For example, in a hypothetical case where computer 501 is designed to provide a recommendation to an end user, this recommendation would typically be communicated from network module 515 of computer 501 through WAN 502 to EUD 503. In this way, EUD 503 can display, or otherwise present, the recommendation to an end user. In some embodiments, EUD 503 may be a client device, such as thin client, heavy client, mainframe computer, desktop computer and so on.

REMOTE SERVER 504 is any computer system that serves at least some data and/or functionality to computer 501. Remote server 504 may be controlled and used by the same entity that operates computer 501. Remote server 504 represents the machine(s) that collect and store helpful and useful data for use by other computers, such as computer 501. For example, in a hypothetical case where computer 501 is designed and programmed to provide a recommendation based on historical data, then this historical data may be provided to computer 501 from remote database 530 of remote server 504.

PUBLIC CLOUD 505 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 505 is performed by the computer hardware and/or software of cloud orchestration module 541. The computing resources provided by public cloud 505 are typically implemented by virtual computing environments that run on various computers making up the computers of host physical machine set 542, which is the universe of physical computers in and/or available to public cloud 505. The virtual computing environments (VCEs) typically take the form of virtual machines from virtual machine set 543 and/or containers from container set 544. It is understood that these VCEs may be stored as images and may be transferred among and between the various physical machine hosts, either as images or after instantiation of the VCE. Cloud orchestration module 541 manages the transfer and storage of images, deploys new instantiations of VCEs and manages active instantiations of VCE deployments. Gateway 540 is the collection of computer software, hardware, and firmware that allows public cloud 505 to communicate through WAN 502.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 506 is similar to public cloud 505, except that the computing resources are only available for use by a single enterprise. While private cloud 506 is depicted as being in communication with WAN 502, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 505 and private cloud 506 are both part of a larger hybrid cloud.

### CONCLUSION

The descriptions of the various embodiments of the present teachings have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

While the foregoing has described what are considered to be the best state and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications, and variations that fall within the true scope of the present teachings.

The components, steps, features, objects, benefits, and advantages that have been discussed herein are merely illustrative. None of them, nor the discussions relating to them, are intended to limit the scope of protection. While various advantages have been discussed herein, it will be understood that not all embodiments necessarily include all advantages. Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

Numerous other embodiments are also contemplated. These include embodiments that have fewer, additional, and/or different components, steps, features, objects, benefits and advantages. These also include embodiments in which the components and/or steps are arranged and/or ordered differently.

Aspects of the present disclosure are described herein with reference to a flowchart illustration and/or block diagram of a method, apparatus (systems), and computer program products according to embodiments of the present disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of an appropriately configured computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The call-flow, flowchart, and block diagrams in the figures herein illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or conduct combinations of special purpose hardware and computer instructions.

While the foregoing has been described in conjunction with exemplary embodiments, it is understood that the term “exemplary” is merely meant as an example, rather than the best or optimal. Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments have more features than are expressly recited in each claim. Rather, as the following claims reflect, the inventive subject matter lies in less than all features of a single disclosed embodiment. Thus, the following claims are hereby incorporated into the Detailed Description, with each claim standing on its own as a separately claimed subject matter.

What is claimed is:

1. A structure for sensing material movement, comprising:
a set of first conductive electrodes in a top substrate;
a set of second conductive electrodes in a bottom substrate positioned adjacent to and spaced apart from the set of first conductive electrodes in the top substrate;
a microfluidic chamber defined within a space between the top substrate and the bottom substrate;
a first set of through vias in the top substrate connecting the set of first conductive electrodes to a set of first signal lines on a top side of the top substrate; and
a second set of through vias in the bottom substrate connecting the set of second conductive electrodes to a set of second signal lines on a bottom side of the bottom substrate.

2. The structure of claim 1, further comprising a set of one or more dielectric materials between the top substrate and the bottom substrate.

3. The structure of claim 1, wherein the top substrate is parallel to the bottom substrate.

4. The structure of claim 1, wherein the first set of conductive electrodes of the top substrate and the second set of conductive electrodes of the bottom substrate form a one or more dimensional array of electrodes.

5. The structure of claim 1, wherein the top substrate and the bottom substrate are electrically insulating substrates.

6. The structure of claim 5, wherein the electrically insulating substrates are one of glass, plastic, or organic printed circuit boards.

7. The structure of claim 1, wherein the first set of conductive electrodes and the second set of conductive electrodes are contacted by signal probe lines passing through the top substrate and the bottom substrate, respectively.

8. The structure of claim 1, wherein a capacitance of the microfluidic channel is measured between opposing ones of the first set of electrodes and the second set of electrodes.

9. The structure of claim 8, wherein the first and second sets of conductive electrodes are configured to provide a signal to a deep learning model for identifying movement within a plant cell disposed within the microfluidic chamber based on changes in the capacitance of the microfluidic channel.

10. The structure of claim 1, wherein each conductive electrode of the first set of electrodes and the second set of electrodes are electrically insulated from each other.

11. The structure of claim 1, each conductive electrode of the first set of conductive electrodes and the second set of conductive electrodes are formed from transparent conductive films.

12. The structure of claim 11, wherein the transparent conductive films are indium tin oxide (ITO), fluorine doped tine oxide (FTO), or doped zinc oxide.

13. The structure of claim 1, further comprising a heating element disposed along at least a portion of a bottom surface of the top substrate and at least a portion of a top surface of the bottom substrate.

14. The structure of claim 1, further comprising an illumination device operable to deliver a user-selectable wavelength of light into the microfluidic chamber.

15. The structure of claim 1, further comprising a gasket disposed between the top substrate and the bottom substrate, the gasket defining the microfluidic chamber.

16. The structure of claim 15, further comprising:
an inlet port fluidly communicating with the microfluidic chamber; and
an outlet port fluidly communicating with the microfluidic chamber.

17. A structure for sensing material movement, comprising:
a set of first conductive electrodes in a top substrate;
a set of second conductive electrodes in a bottom substrate positioned adjacent to and spaced apart from the set of first conductive electrodes in a top substrate;
a microfluidic chamber defined within a space between the top substrate and the bottom substrate;
a first set of through vias in the top substrate connecting the set of first conductive electrodes to a set of first signal lines on a top side of the top substrate;
a second set of through vias in the bottom substrate connecting the set of second conductive electrodes to a set of second signal lines on a bottom side of the bottom substrate;
a heating element disposed along at least a portion of a bottom surface of the top substrate and at least a portion of a top surface of the bottom substrate; and
an illumination device operable to deliver a user-selectable wavelength of light into the microfluidic chamber.

18. The structure of claim 17, wherein the first set of conductive electrodes and the second set of conductive electrodes are contacted by signal probe lines passing through the top substrate and the bottom substrate, respectively.

19. The structure of claim 17, wherein a capacitance of the microfluidic channel is measured between opposing ones of the first set of electrodes and the second set of electrodes.

20. A method for sensing movement within plant cells, comprising:
providing a solution including the plant cells into the microfluidic chamber of the structure of claim 1;
measuring capacitance of the microfluidic channel between opposing ones of the first set of electrodes and the second set of electrodes; and
correlating changes in the capacitance to a movement within plant cells.
